(19) United States
(12) Patent Application Publication
(10) Pub. No.: US 2011/0117017 A1
(43) Pub. Date: May 19, 2011

(54) PEPTIDES WITH PROPERTIES OF AN ALLOSTERIC ANTAGONIST SELECTIVE FOR THE ALPHA 1A ADRENERGIC RECEPTOR AND USES THEREOF

(76) Inventors: Nicolas Gilles, Mitry Mory (FR); André Menez, Magny-Les-Hameaux (FR); Renée Menez, Magny-Les-Hameaux (FR); Christopher Menez, Paris (FR); Cécile Berlioz, Toulouse (FR)

(21) Appl. No.: 12/279,250

(22) PCT Filed: Feb. 23, 2007
(86) PCT No.: PCT/FR2007/000325

§ 371 Date: Oct. 29, 2009

(30) Foreign Application Priority Data
Feb. 23, 2006 (FR) 0601584

(51) Int. Cl. A61K 38/17; C07K 14/46; C07K 14/72; C07H 21/04; A61P 13/02; A61P 15/10; A61P 35/00; A61P 9/12; C40B 30/04; G01N 33/566; C12N 15/63; C12N 5/10; C12N 1/00; A61K 49/00

(52) U.S. Cl. 424/9.1; 530/324; 514/21.3; 530/402; 536/23.5; 506/9; 435/7.21; 435/320.1; 435/325; 435/243; 435/419

(57) ABSTRACT

Peptides characterized by: a) a sequence selected from the group consisting of the sequence SEQ ID NO: 2, and the derived variants having at least 70% identity or 80% similarity with the entire sequence SEQ ID NO: 2, b) a three-finger structure including eight cysteine residues linked by four disulfide bridges, respectively between the first and the third cysteine, the second and the fourth cysteine, the fifth and the sixth cysteine, and the seventh and the eighth cysteine, and c) an activity of an allosteric antagonist selective for the alpha 1a adrenergic receptor, and therapeutic and pharmacological uses thereof.

The present invention relates to peptides having an activity of an allosteric antagonist selective for the alpha1a adrenergic receptor and to therapeutic and pharmacological uses thereof.

Conventional adrenergic receptors (adrenoreceptors or AR) group together nine pharmacologically characterized receptor subtypes: three alpha1 (α₁) adrenoreceptors: alpha1a (α₁ₐ)/alpha1b (α_{1b}), alpha1d (α_{1d}); three alpha2 (α₂) adrenoreceptors: alpha2a (α₂ₐ), alpha1b (α_{2b}), alpha2c (α_{2c}) and three beta (β) adrenoreceptors: beta1 (β₁), beta2 (β₂), beta3 (β₃). These nine receptors, which are all activated by catecholamines, adrenalin and noradrenalin, produce varied physiological effects, through signal transduction by coupling to a G protein.

The alpha1 receptors are especially expressed in the post-synaptic position of the sympathetic nervous system and their activation leads to contraction of smooth muscles which are under their control. These receptors play a major role in the cardiovascular and urogenital system, in the normal or pathological state (for a review see: Piascik et al., Pharmacol. Ther., 1996, 72, 215-241; Michelotti et al., Pharmacology & Therapeutics, 2000, 88, 281-309). In addition, it has been demonstrated that the muscle tonicity of all the urogenital organs in men and in women is principally controlled by activation of the alpha1a subtype, whereas that of the veins and arteries is especially controlled by activation of the alpha1b and alpha1d subtypes (Barrow J. C. et al., J. Med. Chem., 2000, 43, 2703-2718).

Consequently, blocking the alpha1a receptors makes it possible to obtain relaxation of the urogenital tract smooth muscles which can be used in the treatment of urinary dysfunctions and erectile disorders (Moreland et al., The Journal of Pharmacology & Experimental Therapeutics, 2004, 308, 797-804; Guiliano et al., Progrès en Urologie [Progress in Urology], 1997, 7, 24-33). The main pathologies which can be treated are:

Functional obstructions of the urinary tract in women or in men. Prostate adenoma, or benign prostate hyperplasia (BPH), corresponds to the normal change in the size of the prostate, which increases from 40 years of age. This hypertrophy of the prostate, the frequency of which increases with age (80% of men over the age of 70) can be accompanied by a more or less substantial obstruction of the urethra, responsible for urinary problems.

Various approaches have been envisioned for treating this condition; each time, it involves blocking the functioning of an enzyme (5-a-reductase), of a transporter (NET), or of a receptor sensitive to serotonin, to cannabinoid, to glutamate, to calcium or to adrenalin. However, the best results have been obtained by blocking alpha1 adrenergic receptors. This is because blocking these receptors allows smooth muscle relaxation and normal urine flow;

Incontinence: incontinence may be due to pressure from the bladder that is greater than the retention strength of the urethra. Blocking the alpha1a adrenoreceptors would make it possible to reduce the bladder pressure;

Erectile disorders: certain erectile problems can be treated by inhibiting the activity of alpha1a adrenoreceptors, in order to promote irrigation of the corpora cavernosa.

In addition, it has been suggested that blocking the alpha1a adrenoreceptors would have a preventive and curative effect on prostate tumors (European patents EP 0 799 618 and EP 0 799 619, Thebault et al., The J. Clin. Invest., 2003, 111, 1691-1701).

Two classes of molecules are currently available for decreasing the activity of alpha1a adrenoreceptors in the urogenital organs:

### —Alpha1 Adrenergic Receptor Antagonists

Numerous competitive adrenalin antagonists which bind specifically to the orthosteric site of alpha1 adrenergic receptors have been identified: quinazolines (prazosin, tetrazosin, alfusozin, doxazocin), piperazines (RWJ-38063, RWJ-68141, RWJ-68157, RWJ-69736), phenylalkylamines (tamsulosin, indoramin), and silodosin. However, all these molecules, with the exception of KMD3213 (silodosin), cause adverse side effects (hypotension), due to the fact they do not possess any selectivity for the alpha1a adrenergic subtype. KMD3213 (silodosin) is the first competitive antagonist which has selectivity for the alpha1a subtype (Shibata et al., Mol. Pharmacol., 1995, 48-250-258). This molecule is in the clinical phase for the treatment of prostate hypertrophy (Drugs, R. D., 2004, 5, 50-51).

### —Allosteric Modulators of the Alpha1 Adrenergic Receptor

The only allosteric modulators currently known for these adrenoreceptors are amilorides (Leppik et al., Mol. Pharmacol., 2000, 57, 436-445) and rho-conotoxins (ρ-conotoxins), represented by the peptide p-TIA (Sharpe et al., Nature Neuroscience, 2001, 4, 902-907; European patent EP 1 117 681). Amilorides are not very specific and active at very high concentrations. The ρ-TIA peptide is a natural peptide of 19 residues, crosslinked by two disulfide bridges (FNRWRCCLIPACRRNHKKFC; SEQ ID NO. 1), extracted from the venom of the marine cone snail Conus tulipa. This peptide has an affinity of the order of 100 nM for al adrenoreceptors, and weak selectivity (affinity of 10 nM) for the alb subtype which controls vessel tonicity. European patent EP 1 117 681 envisions the use of the p-TIA peptide for the prevention and treatment of cardiovascular (hypertension) and urinary (prostate hypertrophy) pathologies, pain and inflammation.

These two allosteric modulators of alpha1 adrenergic receptors are liable to produce adverse side effects, in particular with respect to the vessels (hypotension), due to their absence of selectivity for the alpha1a subtype.

Numerous neurotoxins have been isolated from the venom of the African mambas Dendroaspis angusticeps (green mamba) and Dendroaspis polylepis (black mamba) (for a review see Bradley, N; Pharmacology & Therapeutics, 2000, 85, 87-109; Jolkkonen M. et al., Eur. J. Biochem., 1995, 234, 2, 579-85). The toxins of the “three-finger toxin” family or “cholinergic toxin” family are peptides of 63 to 66 amino acids having four disulfide bridges (between cysteines 1 and 3, 2 and 4, 5 and 6 and 7 and 8: bridges 1-3, 2-4, 5-6 and 7-8) and a characteristic three-finger structure in which loops I, II and III form the three central fingers of the hand and the disulfide bridges form the palm of the hand. These toxins are divided up into several groups according to their activity: muscarinic toxins (MT) which bind to muscarinic-type acetylcholine receptors, alpha-neurotoxins (α-neurotoxins) which bind to nicotinic-type post-synaptic acetylcholine receptors, and fasciculins, which are noncompetitive acetylcholinesterase inhibitors. In addition, the phylogenetic study of the sequences of these toxins (Fry et al., J. Mol., Evol., 2003, 57, 110-129) shows that these various functional groups, and in particular the muscarinic toxins and the alpha-neurotoxins (PCT International Application WO 99/24055), correspond to distinct peptide sequences.

Six muscarinic toxins have been isolated in Dendroaspis angusticeps [MTX1 (MT1), MTX2 (MT2), MTX3 (MT3 or m4-tox), MTX4 (MT4), MTX5 (MT5), MTX7 (MT7, ml-tox)], and two in Dendroaspis polyepsis [MT-alpha (MTα) and MT-beta (mTβ)]. Despite a strong sequence homology, these peptides have a specificity in terms of their interactions with the various muscarinic receptor subtypes and distinct pharmacological effects.

Due to this specificity, these peptides have been used as a tool for determining the physiological role of certain muscarinic receptor subtypes.

The inventors have isolated a new toxin from Dendroaspis angusticeps venom. This toxin, called AdTx1, selectively binds the alpha1a adrenergic receptor and allosterically decreases the affinity of the orthosteric ligands.

AdTx1 has a sequence of 65 amino acids (SEQ ID NO. 2): LTC₁VTSKSIFGITTEDC₂PDGQNLC₃FKRRHYVVPKIYDSTRGC₄AATC₅PIPENY DSIHC₆C₇KTDKC₈NE.

The structure of AdTx1 comprises 4 disulfide bridges, between the cysteines at positions 3 and 24 (cysteines 1 and 3: bridge 1-3), 17 and 42 (cysteines 2 and 4: bridge 2-4), 46 and 57 (cysteines 5 and 6: bridge 5-6), and 58 and 63 (cysteines 7 and 8: bridge 7-8), characteristic of the three-finger toxin family which acts on the cholinergic system. The sequence of AdTx1 shows very strong homology with certain sequences of the group of muscarinic toxins in the three-finger toxin family:

TABLE I
Homology between AdTx1 and certain three-
finger muscarinic toxins

| | Number | | | |
|---|---|---|---|---|
| | of amino | SwissProt | | |
| Peptide | acids | accession No. | Identity | Similarity |
| MT-beta | 65 | P80495 | 96% (63/65) | 96% (63/65) |
| Synergistic- | 65 | P25518 | 95% (61/65) | 96% (62/65) |
| like venom | | | | |
| protein CM-3 | | | | |
| MT3 | 65 | P81031 | 81% (53/65) | 89% (58/65) |
| Muscarinic | 65 | P82463 | 76% (49/65) | 79% (51/65) |
| toxin-like | | | | |
| protein 2 | | | | |
| (MTLP-2) | | | | |
| MT4 | 66 | Q9PSN1 | 74% (49/66) | 86% (57/66) |
| MT2 | 65 | P18328 | 70% (46/75) | 80% (52/65) |
| MT1 | 66 | P81030 | 72% (48/66) | 84% (56/66) |
| MT7 | 65 | Q8QGR0 | 67% (44/65) | 78% (51/65) |
| MT-alpha | 66 | P80494 | 71% (47/66) | 83% (55/66) |

This new toxin defines a subgroup of peptides of the three-finger muscarinic toxin group, characterized by their property of an allosteric antagonist of the alpha1a adrenergic receptor. In addition, this property of an allosteric antagonist of the alpha1a adrenergic receptor is new and does not follow obviously from the properties of the three-finger muscarinic toxins which are described in the prior art.

Consequently, the present invention relates to peptides for use as a medicament, said peptides being characterized by:

a) a sequence selected from the group consisting of the sequence SEQ ID NO. 2, and the derived variants having at least 70% identity or 80% similarity with the entire sequence SEQ ID NO. 2,
b) a three-finger structure including eight cysteine residues (cysteines 1 to 8) linked by four disulfide bridges, respectively between cysteines 1 and 3, 2 and 4, 5 and 6, and 7 and 8 (bridges 1-3, 2-4, 5-6 and 7-8), and
c) an activity of an allosteric antagonist selective for the alpha1a (α₁ₐ) adrenergic receptor.

The peptides as defined in the present invention are capable of specifically blocking alpha1a adrenergic receptors due to their selectivity for the alpha1a adrenergic subtype. In addition, unlike inhibitors (competitive antagonists), they act as modulators of agonist affinity; such modulators have the advantage of not blocking the function of the receptor, but only of modulating the responses of said receptors when they are activated by their natural agonist. This modulation appears to be easier to control than with competitive antagonists. Furthermore, there is no plateau effect during massive use of allosteric antagonists, which decreases the potential toxic effects accordingly.

### DEFINITIONS

For the purpose of the present invention, the term “three-finger structure” is intended to mean the characteristic structure of the family of three-finger toxins as defined above, which structure comprises three loops (loops I, II, III) maintained by four disulfide bridges (bridges 1-3, 2-4, 5-6, 7-8);

The identity of a sequence relative to the sequence of SEQ ID NO. 2 as reference sequence is assessed according to the percentage of amino acid residues which are identical, when the two sequences are aligned, so as to obtain the maximum correspondence between them.

The percentage identity can be calculated by those skilled in the art using a sequence comparison computer program such as, for example, that of the BLAST series (Altschul et al., NAR, 1997, 25, 3389-3402).

The BLAST programs are used on the comparison window consisting of the entire SEQ ID NO. 2, indicated as reference sequence.

A peptide which has an amino acid sequence having at least X % identity with a reference sequence is defined, in the present invention, as a peptide whose sequence can include up to 100-X alterations per 100 amino acids of the reference sequence, while at the same time conserving the functional properties of said reference peptide, in the case in point its activity of an antagonist selective for the alpha1a adrenergic subtype. For the purpose of the present invention, the term “alteration” includes consecutive or dispersed deletions, substitutions or insertions of amino acids in the reference sequence. This definition applies by analogy to the nucleotide sequences.

The similarity of a sequence relative to a reference sequence is assessed according to the percentage of amino acid residues which are identical or which differ by virtue of conservative substitutions, when the two sequences are aligned so as to obtain the maximum correspondence between them. For the purpose of the present invention, the term “conservative substitution” is intended to mean the substitution of one amino acid with another which has similar chemical or physical properties (size, charge or polarity), which generally does not modify the functional properties of the peptide.

A peptide which has an amino acid sequence having at least X % similarity with a reference sequence is defined, in the present invention, as a peptide whose sequence can include up to 100-X nonconservative alterations per 100 amino acids of the reference sequence. For the purpose of the present invention, the term “nonconservative alterations” includes consecutive or disperse deletions, nonconservative substitutions or insertions of amino acids in the reference sequence.

For the purpose of the present invention, the expression “allosteric antagonist selective for the alpha1a adrenergic receptor” is intended to mean a peptide which selectively binds the alpha1a adrenergic receptor and is capable of allosterically decreasing the affinity of the orthosteric ligands for said receptor.

According to conventional nomenclature, the orthosteric site is the binding site for the endogenous agonist of the receptor (adrenalin in the case of the alpha1a adrenergic receptor). This site is also the binding site for certain antagonists (prazosin in the case of the alpha1a adrenergic receptor). Allosteric modulation implies that the receptor is capable of binding two ligands, concomitantly, by means of two topographically distinct binding sites; the orthosteric ligand binds to the orthosteric site, whereas the modulator binds to a distinct site (allosteric site). The two binding sites are conformationally linked, to such an extent that the binding of a ligand to site 1 disturbs the structure of site 2, thus modifying its affinity for its own ligand.

In the case of the modulatory peptides according to the invention, the binding of the peptide to the alpha1a adrenergic receptor decreases the affinity of specific antagonists for the receptor, such as prazosin, and vice versa.

The activity of an allosteric antagonist selective for the alpha1a adrenergic receptor can be demonstrated by any conventional technique known to those skilled in the art:

by measuring the binding of an orthosteric ligand (prazosin) in the presence of the allosteric ligand (peptide), by means of a conventional ligand/receptor binding assay; the displacement of the binding of the orthosteric ligand to the alpha1a adrenergic receptor, by increasing concentrations of peptide, demonstrates that the peptide is an inhibitor of the alpha1a adrenergic receptor; the absence of complete displacement indicates an allosteric modulation. The selectivity for the alpha1 adrenergic receptor is demonstrated by binding assays, in the presence of the other adrenergic receptor subtypes (alpha1b, alpha1d) and types (alpha2, beta);

by measuring the kinetics of dissociation of the labeled orthosteric ligand/receptor complexes, in the presence of the allosteric ligand, according to the principle described in Ellis J. and Seidenberg M, Mol. Pharmacol., 2000, 58: 1451-1460;

by measuring the inhibition of the activation of the alpha1a receptors expressed in eukaryotic cells, in order to show its antagonistic nature. Eukaryotic cells of COS or HEK type, for example, expressing an adrenergic receptor, for example alpha1a, have the ability to release calcium into the cytosole during activation of the receptor, by adrenalin for example. Thus, when adrenalin binds to its orthosteric site, the receptor is activated. It changes conformation in order to be able to bind a cytoplasmic G protein. This binding induces a cascade of events allowing, inter alia, the synthesis of diacylglycerol and of inositol triphosphate (IP3). The latter, by binding to the IP3 receptor, allows the release of calcium in the cytosole. It is this variation in calcium concentration which is followed by fluorescence. This technique makes it possible to show the agonistic or antagonistic nature of a product.

The invention encompasses the use of natural, synthetic or recombinant peptides having an activity of an allosteric antagonist selective for the alpha1a adrenergic receptor.

The invention encompasses in particular the use of variants obtained by mutation (insertion, deletion, substitution) of one or more amino acids in the sequence SEQ ID NO. 2 as long as said variant conserves an activity of an allosteric antagonist selective for the alpha1a adrenergic receptor.

The invention also encompasses the use of modified peptides derived from the above peptides by introduction of any modification at the level of an amino acid residue or residues, of the peptide bond or of the ends of the peptides, as long as said modified peptide conserves an activity of an allosteric antagonist selective for the alpha1a adrenergic receptor. These modifications which are introduced into the peptides by the conventional methods known to those skilled in the art, include, in a nonlimiting manner: the substitution of a natural amino acid with a nonproteinogenic amino acid (D amino acid or amino acid analog); the addition of a chemical group (lipid, oligosaccharide or polysaccharide) to a reactive function, in particular the side chain R; the modification of the peptide bond (—CO—NH—), in particular with a bond of the retro or retro-inverso type (—NH—CO—) or a bond different from the peptide bond; cyclization; fusion of the sequence of said peptide with that of a peptide or of a protein of interest (epitope of interest for immunodetection); tags (biotin, peptides, flag, in particular) that can be used to purify the peptide, in particular in a form that can be cleaved by a protease, fluorescent protein; coupling to a suitable molecule, in particular a label, for example a fluorochrome. These modifications are intended in particular to increase the stability and more particularly the resistance to proteolysis, and also the solubility, or to facilitate the purification or the detection, either of the peptide according to the invention or of alpha1 adrenergic receptors.

For medical uses, the peptide is advantageously modified by means well-known to those skilled in the art, in order to change its physiological properties, and in particular in order to improve its ½-life time in the organism (glycosylation: HAUBNER R. et al., J. Nucl. Med., 2001, 42, 326-36; conjugation with PEG: KIM T H. et al., Biomaterials, 2002, 23, 2311-7), its solubility (hybridization with albumin: KOEHLER M F. et al., Bioorg. Med. Chem. Lett., 2002, 12, 2883-6), its resistance to proteases (unnatural amino acids (L conformation, for example)), and/or its intestinal absorption (Lien et al., TIB, 2003, 21, 556-).

The term “natural or synthetic amino acid” is intended to mean the 20 natural α-amino acids commonly found in proteins (A, R, N, D, C, Q, E, G, H, I, L, K, M, F, P, S, T, W, Y and V), certain amino acids rarely encountered in proteins (hydroxyproline, hydroxylysine, methyllysine, dimethyllysine, etc.), amino acids which do not exist in proteins, such as (3-alanine, γ-aminobutyric acid, homocysteine, ornithine, citrulline, canavanine, norleucine, cyclohexylalanine, etc., and also the enantiomers and the diastereoisomers of the above amino acids.

According to an advantageous embodiment of said peptide, it is the MT-beta toxin (SWISSPROT P80495, SEQ ID NO. 3), a natural peptide extracted from the venom of the snake Dendroaspis polylepis (black mamba); the sequences of the MT-beta and AdTx1 toxins differ only at the residues at positions 38 and 43, which are respectively I₃₈ and V₄₃ (MT-beta), and S₃₈ and A_{43 (AdTx}1).

According to another advantageous embodiment of said peptide, cysteine 1 is the first or the second amino acid residue and/or cysteine 8 is the penultimate or last amino acid residue of the sequence of said peptide. This peptide represents a truncated peptide derived from the above peptides by deletion of at least one of the N- and/or C-terminal residues, located upstream of cysteine 1 or downstream of cysteine 8.

The subject of the present invention is also an expression vector for use as a medicament, said vector comprising a polynucleotide encoding a peptide as defined above, under the control of suitable regulatory sequences for transcription and, optionally, for translation.

In accordance with the invention, the sequence of said polynucleotide is that of the cDNA encoding said peptide; it is in particular the sequence SEQ ID NO. 4 encoding AdTx1. Said sequence can advantageously be modified in such a way that the codon usage is optimal in the host in which it is expressed. In addition, said polynucleotide can be linked to at least one heterologous sequence.

The expression “heterologous sequence relative to a nucleic acid sequence encoding a peptide as defined in the present invention” is intended to mean any nucleic acid sequence other than those which, naturally, are immediately adjacent to said nucleic acid sequence encoding said peptide.

In accordance with the invention, said recombinant vector comprises an expression cassette including at least one polynucleotide as defined above, under the control of suitable regulatory sequences for transcription and, optionally, for translation (promoter, enhancer, intron, initiation codon (ATG), stop codon, polyadenylation signal).

Numerous vectors into which a nucleic acid molecule of interest can be inserted in order to introduce it into and maintain it in a eukaryotic or prokaryotic host cell are known in themselves; the choice of a suitable vector depends on the use envisioned for this vector (for example, replication of the sequence of interest, expression of this sequence, maintenance of this sequence in extrachromosomal form, or else integration into the chromosomal material of the host), and also on the nature of the host cell. For example, use may be made, inter alia, of viral vectors such as adenoviruses, retroviruses, lentiviruses, AAVs and baculoviruses, into which the sequence of interest has been inserted beforehand; said sequence (isolated or inserted into a plasmid vector) may also be associated with a substance which allows it to cross the host cell membrane, such as a transporter, for instance a nanotransporter, or a preparation of liposomes or of cationic polymers, or else can be introduced into said host cell by using physical methods such as electroporation or microinjection. In addition, these methods may advantageously be combined, for example by using electroporation combined with liposomes.

A subject of the present invention is also a pharmaceutical composition, characterized in that it comprises at least one peptide, one polynucleotide encoding said peptide or one vector as defined above, and a pharmaceutically acceptable carrier.

The pharmaceutical composition according to the invention is in a galenical form suitable for parenteral (subcutaneous, intramuscular, intravenous), enteral (oral, sublingual) or local (nasal, rectal, vaginal) administration.

The pharmaceutically acceptable carriers are those conventionally used.

A subject of the present invention is also the use of at least one peptide and/or one vector as defined above, for the preparation of a medicament having an activity of an allosteric antagonist selective for the alpha1a (α₁ₐ) adrenergic receptor, for use in the treatment of a urogenital or cardiovascular pathology, or else of a cancer.

The urogenital pathologies comprise urinary dysfunctions: incontinence, functional obstruction of the urinary tract in women or in men, and erectile disorders.

Preferably, said urogenital pathology is benign prostate hyperplasia; the increase in volume of the prostate results in an obstruction of the urethra, responsible for urinary dysfunctions. The blocking of alpha1a adrenoreceptors allows relaxation of the smooth muscles and a normal urine flow.

The cardiac pathologies comprise mainly arterial hypertension, in so far as the blocking of alpha1a receptors leads to hypotension. Certain forms of hypertension are due to a pheochromocytoma; the use of an alpha-blocker is recommended before surgical intervention.

The cancer pathologies comprise mainly prostate cancer, in so far as alpha1a adrenergic receptors are predominantly expressed in this organ. Inhibition of the adrenoreceptors would slow down the proliferation of the epithelial cells of the prostate cancer.

A subject of the present invention is also the use of the peptide of sequence SEQ ID NO. 2 or of a derived variant having an activity of an allosteric antagonist selective for the alpha1a (α₁ₐ) adrenergic receptor as defined above, as a tool for studying the alpha1a adrenergic receptor.

According to an advantageous embodiment of said use, said peptide is coupled to a suitable label.

The peptides as defined in the present invention may be labeled directly or indirectly with a radioactive or nonradioactive compound, by covalent or noncovalent coupling, in order to obtain a detectable and/or quantifiable signal.

The labeling is in particular radioactive, magnetic or fluorescent labeling, carried out according to the methods well-known to those skilled in the art. The directly detectable labels are in particular radioactive isotopes such as tritium (³H) and iodine (¹²⁵I), or luminescent compounds such as radioluminescent, chemiluminescent, bioluminescent, fluorescent or phosphorescent agents. The indirectly detectable labels include in particular biotin and B epitopes.

The labeling is in particular carried out:

by grafting a fluorophore onto a reaction amine, i.e. an amine borne by a lysine. It is, for example, possible to obtain AdTx1 mutated at position 34, the lysine being replaced with an arginine or a homoarginine, and in which the Cy3B™ reagent (Amersham) has been grafted onto one or more of the other lysines of the AdTx1,

by direct incorporation of a fluorophore by chemical synthesis (at the N- or C-terminal),

by incorporation of a reaction group (free cysteine, biotin) by recombinant production or synthesis, and then use of this group to graft a fluorophore.

Such labeled peptides are in particular used to localize the alpha1a adrenergic receptors, in vitro and in vivo, so as to determine their tissue expression profile, under physiological or pathological conditions or in response to an endogenous or exogenous stimulus.

A subject of the present invention is also a method for detecting alpha1a adrenergic receptor(s), in vitro and in vivo, comprising at least the following steps:

bringing cells to be analyzed into contact with a labeled peptide as defined above, and

detecting the labeled cells by any suitable means.

The detection of the receptors, in vivo, in the body of a mammal (cell imaging), in particular in real time, comprises a prior step of administering said peptide to said mammal (parenteral injection, oral administration).

The labeling of the cells is in particular fluorescent labeling or magnetic labeling, detectable by any technique known to those skilled in the art (fluorescence microscopy, flow cytometry, magnetic resonance imaging).

Alternatively, the labeled peptides are used to screen molecule libraries, with the aim of identifying other allosteric ligands for the alpha1a adrenergic receptor.

A subject of the present invention is also a method for screening for allosteric ligands for the alpha1a adrenergic receptor, comprising at least the following steps:

bringing an alpha1a adrenergic receptor, in the presence of a library of test molecules, and a labeled peptide as defined above, and

identifying the molecules capable of displacing the binding of said peptide to said receptor, by any suitable means.

In addition, the complexes between the peptide as defined in the present invention and the alpha1a adrenergic receptor can be advantageously used to obtain crystals; such crystals make it possible to determine the three-dimensional structure of the alpha1a adrenergic receptor, by X-ray diffraction.

A subject of the present invention is also a method for preparing crystals of the alpha1 adrenergic receptor, comprising at least the following steps:

a) bringing the alpha1 adrenergic receptor into contact with a peptide as defined above, so as to form receptor/ligand complexes, and
b) incubating the complexes obtained in a) under conditions and for a period of time sufficient to obtain the formation of crystals.

A subject of the present invention is also a receptor/ligand complex in which the receptor is the alpha1a adrenergic receptor and the peptide is a peptide as defined above, optionally coupled to a suitable label.

A subject of the present invention is also the peptide of sequence SEQ ID NO. 2 and the peptides that are variants of SEQ ID NO. 2 comprising the substitution of the lysine at position 34 of the sequence SEQ ID NO. 2 with another amino acid, in particular an arginine or a homoarginine.

According to an advantageous embodiment of said peptide, it is coupled to a suitable label.

A subject of the present invention is also a polynucleotide, an expression cassette, a recombinant vector and a modified prokaryotic or eukaryotic host cell, derived from the above peptide.

According to an advantageous embodiment of said polynucleotide, it has the sequence SEQ ID NO. 4 encoding AdTx1.

The invention encompasses in particular:

a) expression cassettes comprising at least one polynucleotide as defined above, under the control of suitable regulatory sequences for transcription and, optionally, for translation (promoter, enhancer, intron, initiation codon (ATG), stop codon, polyadenylation signal), and
b) recombinant vectors comprising a polynucleotide in accordance with the invention. Advantageously, these vectors are expression vectors comprising at least one expression cassette as defined above.

The polynucleotides, the recombinant vectors and the transformed cells as defined above can be used in particular for the production of the peptides as defined in the present invention.

The polynucleotides according to the invention are obtained by the conventional methods, known in themselves, according to standard protocols such as those described in Current Protocols in Molecular Biology (Frederick M. AUSUBEL, 2000, Wiley and Son Inc., Library of Congress, USA) and Molecular Cloning: A Laboratory Manual, Third Edition, (Sambrook et al., 2001, Cold Spring Harbor, N.Y.: Cold Spring Harbor Laboratory Press).

For example, they can be obtained by amplification of a nucleic sequence by PCR or RT-PCR, by screening genomic DNA libraries by hybridization with a homologous probe, or else by total or partial chemical synthesis. The recombinant vectors are constructed and introduced into host cells by the conventional recombinant DNA and genetic engineering techniques, which are known in themselves.

The peptides and their derivatives (variants, modified peptides) as defined above are prepared by the conventional techniques known to those skilled in the art, in particular by solid-phase or liquid-phase synthesis or by expression of a recombinant DNA in a suitable cell system (eukaryotic or prokaryotic).

More specifically,

the peptides and their derivatives can be solid-phase synthesized, according to the Fmoc technique, originally described by Merrifield et al. (J. Am. Chem. Soc., 1964, 85: 2149-), and purified by reverse-phase high performance liquid chromatography;

the peptides and their derivatives, such as the variants, can also be produced from the corresponding cDNAs, obtained by any means known to those skilled in the art; the cDNA is cloned into a eukaryotic or prokaryotic expression vector and the protein or the fragment produced in the cells modified with the recombinant vector is purified by any suitable means, in particular by affinity chromatography.

In addition to the above arrangements, the invention also comprises other arrangements, which will emerge from the description which follows, which refers to exemplary embodiments of the subject of the present invention, with reference to the attached drawings in which:

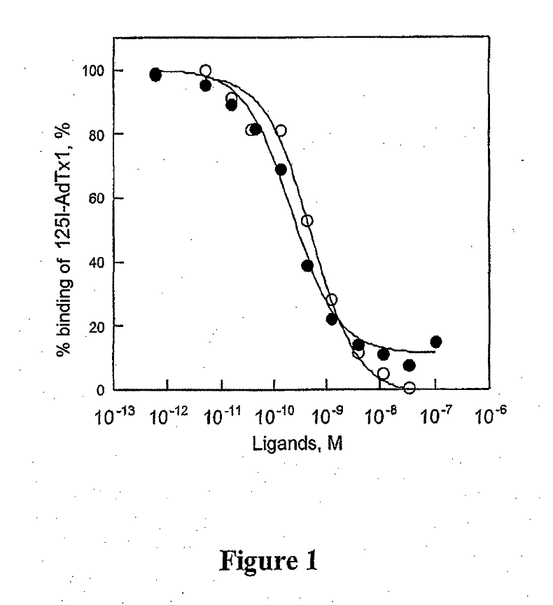

FIG. 1 illustrates the displacement of the binding of the radiolabeled AdTx1 peptide (¹²⁵I-AdTx1) to the alpha1a adrenergic receptor, in the presence of prazosin () or of AdTx1 (∘). The IC₅₀ values are 2.2×10⁻¹⁰ M and 5×10⁻¹⁰ M for prazosin and the AdTx1 peptide, respectively;

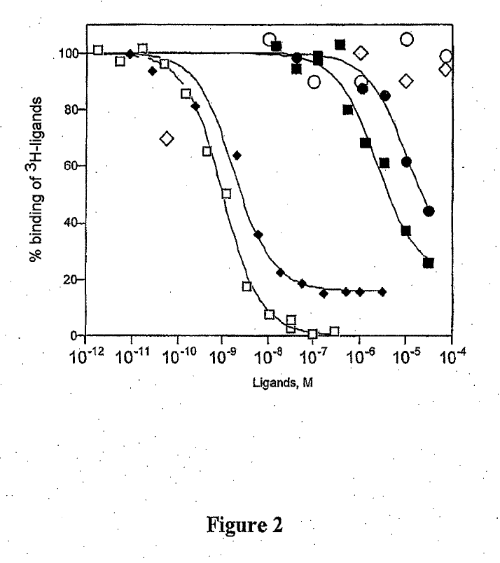

FIG. 2 illustrates the displacement of the binding of ³H-prazosin, ³H-rauwolscine and ³H-CPG12177 to the various adrenergic receptor subtypes, by prazosin or the AdTx1 peptide. (□) displacement, by prazosin, of ³H-prazosin binding to the alpha1a receptor (IC₅₀=0.99×10⁻⁹ M). (♦) displacement by AdTx1, of ³H-prazosin binding to the alpha1a receptor (IC₅₀=1.8×10⁻⁹ M). (▪) displacement, by AdTx1, of ³H-prazosin binding to the alpha1b receptor (IC₅₀=2.3×10⁻⁶ M). () displacement, by AdTx1, of ³H-prazosin binding to the alpha1d receptor (IC₅₀=9.9×10⁻⁶ M). (∘) displacement, by AdTx1, of ³H-rauwolscine binding to the alpha1a receptor (IC₅₀>5×10⁻⁵ M). (⋄) displacement, by AdTx1, of ³H-CGP12177 binding to the beta1 receptor (IC₅₀>5×10⁻⁹ M);

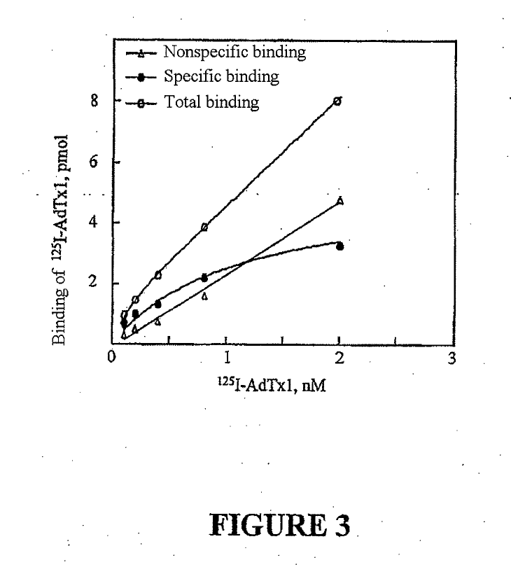

FIG. 3 represents the hot saturation of ¹²⁵I-AdTx1 on yeast membranes expressing the alpha1a adrenergic receptor. Various concentrations of ¹²⁹I-AdTx1 are incubated for 20 h in the presence of 20 μg of yeast membranes. The nonspecific binding is measured in the presence of 1 μm of AdTx1. (Δ) nonspecific binding. () specific binding. (∘) total binding. The specific binding can be saturated and is high-affinity binding, equal to 0.8±0.2 nM;

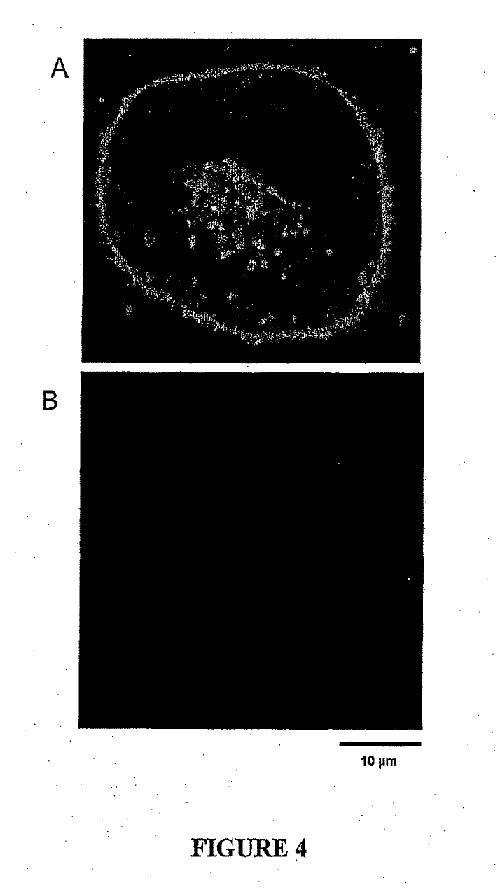

FIG. 4 illustrates the labeling, with the AdTx1-Cy3B™ fluorescent derivative, of the h-alpha1a (A) and h-alpha1b (B) adrenergic receptors transiently expressed in COS cells.

### EXAMPLE 1

### Preparation of the Adtx1 Polypeptide

### 1) Chemical Synthesis

The AdTx1 peptide is solid-phase synthesized by the Fmoc (fluorenyl methyloxy carbonyl) technique, using dicyclohexylcarbodiimide/1-hydroxy-7-azabenzotriazole (HOAT) as coupling agent and N-methylpyrrolidone as solvent (Mourier et al., Molecular Pharmacology, 2003, 63, 26-35). Briefly, the synthesis is carried out from the C-terminal end to the N-terminal end of the peptide using 0.05 mmol of resin. At the end of the synthesis, the resin/peptide is treated with a mixture of 9 ml of trifluoroacetic acid, 0.5 ml of triisopropylsilane and 0.5 ml of distilled water. The peptide is then cleaved from the resin after two hours of incubation. The mixture is filtered over cold ethyl ether and centrifuged twice. The precipitate thus obtained is dissolved in a solution of acetic acid at 10% and lyophilized. The reduced synthetic toxin is purified by reverse-phase chromatography (HPLC) on a Discovery® Bio Wide Pore C5, 25 cm×10 mm, 10 μm semipreparative column (SUPELCO) with a gradient of 40% to 70% of solvent B in 150 minutes (A: 0.1% TFA, B: 50% acetonitrile and 0.1% TFA), with a flow rate of 4.5 ml/min. The detection is followed at 220 nm.

The synthetic toxin is then folded in 100 mM Tris buffer, pH 8.0, in the presence of reduced glutathione (GSSG) and oxidized glutathione (GSH) with a GSSG/GSH molar ratio of 1/1 and a concentration of 1 mM. After three days at 4° C., in the dark and under argon, the folded synthetic toxin is purified by reverse-phase chromatography (HPLC) under the same conditions as described above. The concentration of synthetic toxin is 5 μM.

### 2) Production of Recombinant Polypeptide

The cloning of the nucleotide sequence encoding AdTx1 is carried out by homologous recombination according to the technology (Gateway®, Invitrogen). A polynucleotide fragment (SEQ ID NO. 4) comprising successively from 5′ to 3′: an attB1 recombination sequence, the TEV cleavage site (ENLYFQG), the sequence encoding AdTx1 (SEQ ID NO. 3), a pseudo stop, the sequence encoding the Stag peptide, a stop codon, and the attB2 recombination sequence, was amplified by PCR. The PCR product was cloned by homologous recombination into the donor plasmid pDONR221 (Invitrogen). The clone thus obtained is used to generate recombinant expression vectors suitable for the expression of AdTx1 in a suitable cell system.

### EXAMPLE 2

### Analysis of AdTx1 Binding to the Alpha1 Adrenergic Receptors

### 1) Materials and Methods

### a) Iodination of the AdTx1 Toxin

The iodination of AdTx1 is carried out by means of a halogenation reaction, catalyzed by lactoperoxidase.

The reaction mixture containing 50 μl of 0.1 M phosphate buffer, at pH 7.3, 10 μl of 100 μM toxin, 10 μl of 1/50,000 H₂O₂, 1 mCi [¹²⁵I] and 0.7 unit of lactoperoxidase (Sigma) is incubated for 1 minute at 25° C. The iodinated toxin is then purified by reverse-phase HPLC as previously described (Krimm I. et al., J. Mol. Biol., 1999, 285, 1749-63).

### b) Preparation of Membranes Containing the Adrenergic Receptor

Each receptor subtype, α₁ₐα_{1b}, α_{1d}, α₂ₐ and β₁, is expressed in the yeast Pichia pastoris, transformed with an expression plasmid comprising the cDNA corresponding to said receptor. Each clone is cultured in the same manner. The Pichia pastoris clones are inoculated into 10 ml of medium (1% yeast extract, 2% peptone, 100 mM potassium phosphate, pH 6, 1.3% nitrogenous yeast base, 1% glycerol), overnight at 30°, and the cultures are then diluted in 100 ml of fresh medium and incubated again for 4 h at 30° C. The cultures are subsequently centrifuged (3000 g, 5 min), resuspended in 500 ml of induction medium (1% yeast extract, 2% peptone, 100 mM potassium phosphate, pH 6, 1.3% nitrogenous yeast base, 0.5% methanol), supplemented with 2.5% DMSO, and incubated for 18 h at 20° C. with shaking (200 rpm). The cultures are harvested (3000 g, 15 min, 4° C.) and resuspended in 3 ml of buffer (50 mM potassium phosphate, pH 7.4, 100 mM NaCl, 5% glycerol, 2 mM EDTA and 1 mM PMSF), cooled in ice. Cold glass beads (2 ml; 400-600 μm acid washed glass beads, Sigma) are added to the suspensions, and the mixture is then subjected to eight cycles of 30 seconds of vortexing, followed by being left to stand in ice. The glass beads and the non-lysed cells are then separated from the lysate by centrifugation (5 min at 3000 g, 4° C.), and the pellet is washed under the same conditions. The supernatant is recovered and centrifuged for 1 h at 20,000 g. The pellet of each preparation is resuspended in a buffer (50 mM Tris pH 8, 120 mM NaCl, 10% glycerol, 1 mM PMSF) using a homogenizer, aliquoted, and conserved at −80° C. until use.

c) Binding assays

5 μg of membranes are mixed with a final concentration of 1 nM of ³H-prazosin ([7-methoxy-3H], Perkin Elmer Life Sciences) or of ³H-rauwolscine, [methyl-3H](Perkin Elmer Life Sciences) or of ³H-CPG12177 (Perkin Elmer Life Sciences) in Tris-HCl buffer, pH 7.2, supplemented with 10 mM of MgCl₂, in a final volume of 200 μl, and the mixture is then incubated for 5 hours at ambient temperature, in the presence of increasing doses of AdTx1 (0.01 nM to 100 μM). The nonspecific binding is measured in the presence of 1 μM of prazosin (Sigma) for the binding with ³H-prazosin, of 1 μM of yohimbine (Sigma) for the binding with ³H-rauwolscine, or of 1 μM of propranolol (Sigma) for the binding with ³H-CPG12177. The reaction is stopped by filtration preceded by dilution of the reaction medium in 2 ml of washing buffer (Tris-HCl, pH 7.2, 10 mM), at 4° C. The filtration is carried out over glass filters (GFC, Whatman) pretreated in 0.3% PEI buffer (polyethyleneimine, Sigma). Two successive and rapid washes are carried out. The filters are dried for one hour at 80° C. and 10 ml of Lipoluma Plus (Lumac LMC) are added thereto. The emissions are detected with a Rockbeta 1211 counter (LKB Wallac), which gives the value of each assay in cpm (counts per minute). The analysis of the results is carried out using the Kaleidograph software (Tools for discovery, Synergy Software, PA, USA).

The analysis of the displacement of the binding to the alpha1a adrenergic receptor by the iodinated AdTx1 peptide (0.1 nM) is carried out according to the same protocol as that used for the tritiated ligands, and the radioactivity is measured with a Multigamma 1261 counter (LKB Wallac).

The analysis of the saturation of the alpha1a adrenergic receptor with AdTx1 is carried out by incubating increasing concentrations of the iodinated peptide in the presence of 20 μg of membranes containing the alpha1a adrenergic receptor, for 20 h, and then measuring the radioactivity as above. The nonspecific binding is measured in the presence of 1 μM of AdTx1.

### 2) Results

The analysis of the binding of the AdTx1 peptide to the various adrenergic receptor subtypes alpha1a, 1b, 1d, 2a and beta1 is given in FIGS. 1, 2 and 3.

The AdTx1 peptide is an alpha1a adrenergic receptor ligand (FIG. 1). The competitive binding assays with orthosteric ligands specific for the various adrenergic receptor subtypes (FIG. 2) indicate that the AdTx1 peptide selectively binds the alpha1a adrenergic subtype. Its affinities for the alpha1a, 1b, 1d, 2a and beta1 subtypes, evaluated through the IC₅₀ value, are respectively 1.8×10⁻⁹ M, 2.3×10⁻⁶ M, 9.9×10⁻⁶ M, >5×10⁻⁵ M, and >5×10⁻⁵ M.

The curve for displacement of prazosin binding to the alpha1a adrenergic receptor, in the presence of the AdTx1 peptide (FIGS. 1 and 2), indicates that the displacement is incomplete, which reflects an allosteric modulation.

The results given in FIGS. 1 and 2 indicate that the AdTx1 peptide specifically binds the alpha1a adrenergic receptor and that it allosterically decreases the affinity for the orthosteric ligands, due to this binding. It is therefore an allosteric antagonist selective for the alpha1a adrenergic receptor.

The alpha1a adrenergic receptor saturation curve (FIG. 3) shows that the specific binding of AdTx1 can be saturated and is high-affinity binding, equal to 0.8±0.2 nM.

### EXAMPLE 3

### Labeling of Alpha1a Adrenergic Receptors with the AdTx1Cy3B™ Fluorescent Derivative

### 1) Materials and Methods

The AdTx1 K34R variant was synthesized as described in example 1 for AdTx1. The AdTx1 K34R variant was then coupled to the Cy3B-mono-NHS-ester fluorophore (AMERSHAM) according to the protocol recommended by the manufacturer. COS cells (ATCC) transfected either with an expression vector for the human alpha1a adrenergic receptor, or with an expression vector for the human alpha1b adrenergic receptor, and transiently expressing this alpha1a or alpha1b receptor, were incubated in the presence of 2 μM of AdTx1-Cy3b for 16 h, and then the fluorescence emitted after laser excitation at 543 nm was analyzed using a fluorescence microscope (Leica TCS SP2, LEICA MICROSYSTEMS), at ×40 magnification.

### 2) Results

FIG. 4 illustrates the specific labeling of the alpha1a adrenergic receptors with the AdTx1 fluorescent derivative. FIG. 4A shows intense labeling of the h-alpha1a adrenergic receptors by the AdTx1 fluorescent derivative. By comparison, FIG. 4B shows the absence of labeling of the h-alpha1b adrenergic receptors (FIG. 4B) by the AdTx1 fluorescent derivative.

1. A peptide for use as a medicament, said peptide being characterized by:
a) a sequence selected from the group consisting of the sequence SEQ ID NO. 2, derived variants of sequence SEQ ID NO. 2 having at least 70% identity, and derived variants of sequence SEQ ID NO. 2 having at least 80% similarity with the entire sequence SEQ ID NO. 2,2
b) a three-finger structure including eight cysteine residues linked by four disulfide bridges, respectively between the first and the third cysteine, the second and the fourth cysteine, the fifth and the sixth cysteine, and the seventh and the eighth cysteine, and
c) an activity of an allosteric antagonist selective for alpha1a (α₁ₐ) adrenergic receptor.

2. The peptide as claimed in claim 1, characterized in that it includes sequence SEQ ID NO. 3.

3. The peptide as claimed in claim 1, characterized in that the first cysteine is at position 1 or 2 and/or the eighth cysteine is at the last or penultimate position of said sequence defined in a).

4. An expression vector for use as a medicament, said vector comprising a polynucleotide encoding a peptide as defined in claim 1, under the control of suitable regulatory sequences for transcription and, optionally, for translation.

5. A pharmaceutical composition, characterized in that it comprises at least one peptide as defined in claim 1 and a pharmaceutically acceptable carrier.

6. The use of at least one peptide as defined in claim 1, for the preparation of a medicament having an activity of an allosteric antagonist selective for the alpha1a adrenergic receptor, for use in the treatment of a urogenital or cardiovascular pathology, or else of a cancer.

7. The use as claimed in claim 6, characterized in that said pathology is selected from the group consisting of benign prostate hyperplasia, urinary incontinence, erectile disorders, arterial hypertension and prostate cancer.

8. The use of the peptide as defined in claim 1, as a tool for studying the alpha1a adrenergic receptor.

9. The use as claimed in claim 8, characterized in that said peptide is a variant of the sequence SEQ ID NO. 2, comprising the substitution of the lysine at position 34 of the sequence SEQ ID NO. 2 with another amino acid.

10. The use as claimed in claim 9, characterized in that the lysine at position 34 is substituted with an arginine or a homoarginine.

11. The use as claimed in claim 8, characterized in that said peptide is coupled to a suitable label.

12. The use as claimed in claim 8, characterized in that said study comprises determining the tissue expression profile of the alpha1a adrenergic receptor.

13. The use of the peptide as defined in claim 8, for screening for allosteric ligands of the alpha1a adrenergic receptor.

14. A method for screening for allosteric ligands of the alpha1a adrenergic receptor, comprising at least the following steps:
bringing an alpha1a adrenergic receptor into contact with a library of test molecules and a labeled peptide as defined in claim 11, and
identifying the molecules capable of displacing the binding of said peptide to said receptor.

15. A method for detecting an alpha1a adrenergic receptor, in vitro and in vivo, comprising at least the following steps:
bringing cells to be analyzed into contact with a labeled peptide as defined in claim 11, and
detecting the labeled cells.

16. A method for preparing crystals of the alpha1 adrenergic receptor, comprising at least the following steps:
a) bringing the alpha1 adrenergic receptor into contact with a peptide as defined in claim 1 to form receptor/ligand complexes, and
b) incubating the complexes obtained in a) under conditions and for a period of time sufficient to obtain the formation of crystals.

17. An isolated peptide, characterized in that it has the sequence SEQ ID NO. 2.

18. An isolated peptide, as defined in claim 9.

19. The peptide as claimed in claim 18, characterized in that it is coupled to a suitable label.

20. An isolated receptor/ligand complex, characterized in that said receptor is the alpha1a adrenergic receptor and said peptide is a peptide as defined in claim 1.

21. A polynucleotide, characterized in that it encodes the peptide as claimed in claim 18.

22. The polynucleotide as claimed in claim 21, characterized in that it includes sequence SEQ ID NO. 4.

23. A recombinant vector, characterized in that it comprises a polynucleotide as claimed in claim 22.

24. A cell modified with the polynucleotide as claimed in claim 21.


SEQUENCE LISTING (5 sequences)

SEQ ID NO: 1 - protein, 19 aa, Conus tulipa
FNWRCCLIPACRRNHKKFC

SEQ ID NO: 2 - protein, 65 aa, Dendroaspis angusticeps
LTCVTSKSIFGITTEDCPDGQNLCFKRRHYVVPKIYDSTRGCAATCPIPENYDSIHCCKTDKCNE

SEQ ID NO: 3 - protein, 65 aa, Dendroaspis polylepis
LTCVTSKSIFGITTEDCPDGQNLCFKRRHYVVPKIYDITRGCVATCPIPENYDSIHCCKTDKCNE

SEQ ID NO: 4 - DNA, 196 bp, Dendroaspis angusticeps
cttaacctgcgtgacgagcaaatctatcttcggcattaccacggaagattgcccggacggccagaacttatgtttcaaacgccgccattacgttgtcccaaagatttacgactctacgcgtggctgcgccgcaacctgcccgattccagagaattatgactctattcattgttgtaaaaccgataaatgtaacgaa

SEQ ID NO: 5 - DNA, 336 bp, artificial sequence
  expression cassette
gggggggggacaagtttgtacaaaaaagcaggcttcgagaacctgtatttccaaggcttaacctgcgtgacgagcaaatctatcttcggcattaccacggaagattgcccggacggccagaacttatgtttcaaacgccgccattacgttgtcccaaagatttacgactctacgcgtggctgcgccgcaacctgcccgattccagagaattatgactctattcattgttgtaaaaccgataaatgtaacgaatagaaagaaaccgctgctgctaaattcgaacgtcagcacatggactcttaaacccagctttcttgtacaaagtggtcccccccc